SEQUENCE LISTING (2 sequences)

SEQ ID NO: 1 - DNA, 20 bp, synthetic construct
  misc_feature 1..20: note = K3 CpG ODN
  source 1..20: mol_type = other DNA
atcgactctcgagcgttctc

SEQ ID NO: 2 - DNA, 60 bp, synthetic construct
  misc_feature 1..60: note = K3 CpG A40 ODN
  source 1..60: mol_type = other DNA
atcgactctcgagcgttctcaaaaaaaaaaaaaaaaaaaaaaaaaaaaaaaaaaaaaaaa

(19) United States
(12) Patent Application Publication
(10) Pub. No.: US 2025/0222108 A1
(43) Pub. Date: Jul. 10, 2025

(54) MEDICAL AGENT INCLUDING ADJUVANT AND COMPOSITE OF PHOTOSENSITIVE MOLECULE AND MOLECULE BINDING TO CANCER-SPECIFIC CELL SURFACE MOLECULE

(71) Applicant: KYOTO UNIVERSITY, Kyoto-shi, Kyoto (JP)

(72) Inventors: Ken TAKAHASHI, Kyoto-shi, Kyoto (JP); Hirokazu OKADA, Kyoto-shi, Kyoto (JP); Hiroaki YAKU, Kyoto-shi, Kyoto (JP); Ken ISHII, Tokyo (JP); Koji KOBIYAMA, Tokyo (JP)

(73) Assignee: KYOTO UNIVERSITY, Kyoto-shi, Kyoto (JP)

(21) Appl. No.: 18/703,103

(22) PCT Filed: Oct. 11, 2022
(86) PCT No.: PCT/JP2022/037841

§ 371 Date: Apr. 19, 2024

(30) Foreign Application Priority Data
Oct. 20, 2021 (JP) 2021-171961

(51) Int. Cl. A61K 41/00; A61K 9/00; A61K 39/00; A61K 39/39; A61K 47/54; A61K 47/68; A61P 35/00

(52) U.S. Cl. CPC A61K 41/0057; A61K 9/0019; A61K 39/39; A61K 47/549; A61K 47/68; A61P 35/00; A61K 2039/54; A61K 2039/55561

(57) ABSTRACT

The present invention provides a medicament that improves antitumor effects.

### TECHNICAL FIELD

The present invention relates to medicaments containing an adjuvant and a complex of a photosensitive molecule and a molecule binding to a cancer-specific cell surface molecule, and the like.

### BACKGROUND ART

The number of cancer patients is many and increasing, and the results of existing treatments such as surgery, chemotherapy, and radiation therapy are not satisfactory. In recent years, cancer immunotherapy has joined these existing treatment methods. Cancer immunity is activated by local tumor antigen release (LTAR) and local innate immune activation (LIIA). In situ vaccines, which aim to stimulate immunity by intervening in the treatment of localized tumor, can be said to be a treatment that artificially enhances LIIA. In situ vaccines are expected to produce strong systemic anti-cancer effects while minimizing side effects, and become a new therapeutic strategy for cancer immunity. However, the mainstream of cancer immunotherapy at present is checkpoint inhibitors, which achieve insufficient response rates and pose problems of serious side effects.

Photoimmunotherapy (PIT) is a treatment method that selectively destroys only tumor cells by administering a drug containing a complex of a cancer antigen-specific antibody and the photosensitive molecule IR700dye and irradiating non-thermal near-infrared light to the tumor (Patent Literature 1). In September 2020, Japan became the first country in the world to receive insurance approval for a photoimmunotherapy that combines the antibody preparation ASP1929 obtained by adding IR700dye to an EGFR antibody cetuximab and a near-infrared laser irradiation device to treat unresectable locally progressive or locally recurrent head and neck cancer. The mechanism of action of PIT is that photosensitive molecules excited by light irradiation change the structure of the cell membrane of cancer cells, and as a result, water molecules flow into the cancer cells due to the osmotic pressure difference and cause cell rupture. Therefore, in PIT, a large amount of cancer antigens that have not undergone heat denaturation are released and a superior LTAR effect can be expected. However, the success rate of photoimmunotherapy, which shows the effect in clinical tests, remains at around 40%, and attempts have been made to combine photoimmunotherapy targeting regulatory T cells and to use immune checkpoint inhibitors in combination.

### CITATION LIST

### Patent Literature

[Patent Literature 1]
JP 2018-528268 A

### SUMMARY OF INVENTION

### Technical Problem

Therefore, the problem of the present invention is to improve antitumor effects.

### Solution to Problem

The present inventors previously reported that they had developed K3-SPG which is a complex of humanized K-type CpG-ODN and schizophyllan, as a TLR9 ligand (WO 2016/152767). This substance is an innate immune-activating adjuvant that has a strong ability to induce type I IFN and the ability to induce antigen-specific cytotoxic T cells, and is therefore expected to be applied in the future to the prevention or treatment of diseases such as viruses and cancer. Therefore, the present inventors used mice transplanted with cancer cells, intratumorally administered a substance for photoimmunotherapy and an adjuvant, and attempted a photoimmunotherapy by irradiating near-infrared light. As a result, they not only almost eliminated the tumor subjected to direct administration, but also successfully shrank the tumor not subjected to administration. These effects were confirmed to be higher than those of photoimmunotherapy or adjuvant administration alone. Furthermore, it was confirmed that intravenous administration of the adjuvant after destruction of tumor by photoimmunotherapy results in selective accumulation of the adjuvant only in the tumor site, despite systemic administration. Furthermore, the mice that achieved complete remission among those that underwent intratumoral administration of the substance for photoimmunotherapy and the adjuvant and the photoimmunotherapy by irradiation of near-infrared light were free from proliferation of cancer cells even when the cancer cells were transplanted again. The present inventors conducted further studies based on these findings, and completed the present invention.

Accordingly, the present invention provides the following.

[1] A medicament for treating cancer, comprising a complex of a photosensitive molecule and a molecule binding to a cancer-specific cell surface molecule, and an adjuvant.
[2] A medicament for treating cancer, comprising a complex of a photosensitive molecule and a molecule binding to a cancer-specific cell surface molecule, wherein the aforementioned medicament is for administration in combination with an adjuvant excluding ligands for Toll-like receptor 3 (TLR3).
[3] A medicament for treating cancer, comprising an adjuvant excluding ligands for Toll-like receptor 3 (TLR3), wherein the aforementioned medicament is for administration in combination with a complex of a photosensitive molecule and a molecule binding to a cancer-specific cell surface molecule.
[4] The medicament of any one of [1] to [3], wherein the adjuvant is a ligand for Toll-like receptor 9 (TLR9).
[5] The medicament of [4], wherein the ligand for Toll-like receptor 9 (TLR9) is a complex of a nucleic acid and a polysaccharide.
[6] The medicament of [5], wherein the nucleic acid is a nucleic acid comprising a type-K CpG oligodeoxynucleotide and polydeoxyadenylic acid.
[7] The medicament of [6], wherein the type-K CpG oligodeoxynucleotide is humanized.
[8] The medicament of [6] or [7], wherein the type-K CpG oligodeoxynucleotide comprises the nucleotide sequence shown in SEQ ID NO: 1.
[9] The medicament of any one of [5] to [8], wherein the polysaccharide is β-glucan.
[10] The medicament of [9], wherein the β-glucan is schizophyllan or lentinan.
[11] The medicament of any one of [1] to [3], wherein the adjuvant is a ligand for STING.
[12] The medicament of [11], wherein the ligand for STING is CGAMP.
[13] The medicament of any one of [1] to [12], wherein the medicament is a kit.
[14] The medicament of [13], wherein the complex of a photosensitive molecule and a molecule binding to a cancer-specific cell surface molecule is administered intratumorally or intravenously, and the adjuvant is administered intratumorally or intravenously.
[15] The medicament of [13], wherein the complex of a photosensitive molecule and a molecule binding to a cancer-specific cell surface molecule, and the adjuvant are each administered intravenously.
[16] A method for treating cancer, comprising administering to a subject an effective amount of a complex of a photosensitive molecule and a molecule binding to a cancer-specific cell surface molecule, and an effective amount of an adjuvant excluding ligands for Toll-like receptor 3 (TLR3).
[17] A complex of a photosensitive molecule and a molecule binding to a cancer-specific cell surface molecule, and an adjuvant excluding ligands for Toll-like receptor 3 (TLR3), for use in the treatment of cancer.
[18] Use of a complex of a photosensitive molecule and a molecule binding to a cancer-specific cell surface molecule, and an adjuvant excluding ligands for Toll-like receptor 3 (TLR3), in the production of a medicament for cancer treatment.
[19] A kit for treating cancer, comprising a complex of a photosensitive molecule and a molecule binding to a cancer-specific cell surface molecule, and an adjuvant excluding ligands for Toll-like receptor 3 (TLR3).
[20] A kit for treating cancer, comprising a complex of a photosensitive molecule and a molecule binding to a cancer-specific cell surface molecule, wherein the aforementioned kit is for administration in combination with an adjuvant excluding ligands for Toll-like receptor 3 (TLR3).
[21] A kit for treating cancer, comprising an adjuvant excluding ligands for Toll-like receptor 3 (TLR3), wherein the aforementioned kit is for administration in combination with a complex of a photosensitive molecule and a molecule binding to a cancer-specific cell surface molecule.
[22] A kit for treating cancer, comprising the complex of a photosensitive molecule and a molecule binding to a cancer-specific cell surface molecule, and an adjuvant, each described in any one of [1] to [12].
[23] The kit of any one of [19] to [22], wherein the adjuvant is a ligand for Toll-like receptor 9 (TLR9).
[24] The kit of [23], wherein the ligand for Toll-like receptor 9 (TLR9) is a complex of a nucleic acid and a polysaccharide.
[25] The kit of [24], wherein the nucleic acid is a nucleic acid comprising a type-K CpG oligodeoxynucleotide and polydeoxyadenylic acid.
[26] The kit of [25], wherein the type-K CpG oligodeoxynucleotide is humanized.
[27] The kit of or [26], wherein the type-K CpG oligodeoxynucleotide comprises the nucleotide sequence shown in SEQ ID NO: 1.
[28] The kit of any one of [24] to [27], wherein the polysaccharide is β-glucan.
[29] The kit of [28], wherein the β-glucan is schizophyllan or lentinan.
[30] The kit of any one of [19] to [22], wherein the adjuvant is a ligand for STING.
[31] The kit of [30], wherein the ligand for STING is cGAMP.

### Advantageous Effects of Invention

The present invention can improve antitumor effects.

### BRIEF DESCRIPTION OF DRAWINGS

FIG. 1 is a diagram showing treatment schedule in each of an untreated group (Ctrl), an adjuvant administration group (K3-SPG), a photoimmunotherapy group (anti-CD44 Ab-IR700), and a combination therapy group (K3-SPG/anti-CD44 Ab-IR700) of mice transplanted with pancreatic cancer cells.

FIG. 2 is a diagram showing changes in the average size of pancreatic cancer (N=6) in each of an untreated group (Ctrl), an adjuvant administration group (K3-SPG), a photoimmunotherapy group (anti-CD44 Ab-IR700), and a combination therapy group (K3-SPG/anti-CD44 Ab-IR700) of mice transplanted with pancreatic cancer cells. The treated side shows the change in the average size of the tumor on the right side of the back, and the opposite side shows the change in the average size of the tumor on the left side of the back.

FIG. 3 is a diagram showing changes in the size of pancreatic cancer of each mouse in each of an untreated group (Ctrl), an adjuvant administration group (K3-SPG), a photoimmunotherapy group (anti-CD44 Ab-IR700), and a combination therapy group (K3-SPG/anti-CD44 Ab-IR700) of mice transplanted with pancreatic cancer cells. The treated side shows the change in the average size of the tumor on the right side of the back, and the opposite side shows the change in the average size of the tumor on the left side of the back.

FIG. 4 is a diagram showing treatment schedule in each of an untreated group (Ctrl), an adjuvant administration group (K3-SPG), a photoimmunotherapy group (anti-CD44 Ab-IR700), and a combination therapy group (K3-SPG/anti-CD44 Ab-IR700) of mice transplanted with colorectal cancer cells.

FIG. 5 is a diagram showing changes in the size of pancreatic cancer of each animal and changes in the average thereof (N=4) in each of an untreated group (Ctrl), an adjuvant administration group (K3-SPG), a photoimmunotherapy group (anti-CD44 Ab-IR700), and a combination therapy group (K3-SPG/anti-CD44 Ab-IR700) of mice transplanted with colorectal cancer cells.

FIG. 6 is a diagram showing the difference in the accumulation of Alexa647-K3-SPG in the left and right tumors on the back when anti-CD44 Ab-IR700 was intratumorally administered to the tumor on the left side of the back of a mouse transplanted with pancreatic cancer cells, and after near-infrared irradiation, Alexa647-K3-SPG was intravenously administered.

FIG. 7 is a diagram showing changes in the average size of pancreatic cancer (N=6) in each of an untreated group (Control), an adjuvant administration group (2′3′-cGAMP), a photoimmunotherapy group (anti-CD44 Ab-IR700), and a combination therapy group (2′3′-cGAMP/anti-CD44 Ab-IR700) of mice transplanted with pancreatic cancer cells.

FIG. 8 is a diagram showing changes in the average size of pancreatic cancer (N=6) in each of an untreated group (Control) and PolyI:C administration group (PolyI:C) of mice transplanted with pancreatic cancer cells.

FIG. 9 is a diagram showing changes in the size of pancreatic cancer in each of an untreated group (Control; N=6), CR group (N=4), and nonCR group (N=2) of mice transplanted again with pancreatic cancer cells.

### DESCRIPTION OF EMBODIMENTS

### 1. Medicament

The present invention provides a medicament for treating cancer, containing a complex of a photosensitive molecule and a molecule binding to a cancer-specific cell surface molecule, and an adjuvant (hereinafter the medicament (I) of the present invention).

Also, the present invention provides a medicament for treating cancer, containing a complex of a photosensitive molecule and a molecule binding to a cancer-specific cell surface molecule (hereinafter medicament (Ia) of the present invention), which can be used in combination with an adjuvant. Also, the present invention provides a medicament for treating cancer, containing an adjuvant (hereinafter medicament (Ib) the present invention), which can be used in combination with a complex of a photosensitive molecule and a molecule binding to a cancer-specific cell surface molecule. The embodiment of the combined use of a complex of a photosensitive molecule and a molecule binding to a cancer-specific cell surface molecule, and an adjuvant is not limited, and those of ordinary skill in the art (e.g., doctor) can perform in various embodiments according to the kind of the target cancer, treatment stage, and the like.

### (1) Complex of a Photosensitive Molecule and a Molecule Binding to a Cancer-Specific Cell Surface Molecule

The medicament (I) of the present invention contains a complex of a photosensitive molecule and a molecule binding to a cancer-specific cell surface molecule (hereinafter the complex of a binding molecule and a photosensitive molecule of the present invention). The complex of a binding molecule and a photosensitive molecule of the present invention can be produced according to a conventionally-known means. For example, a complex can be formed by linking a molecule binding to a cancer-specific cell surface molecule to a photosensitive molecule via a suitable bond. Bonds include electrostatic bonds, van der Waals bonds, hydrogen bonds, noncovalent bonds such as hydrophobic interactions, covalent bonds such as amide bonds and the like, and the like.

The complex of a binding molecule and a photosensitive molecule of the present invention may contain one or more of the same molecule. In one embodiment, the complex of a binding molecule and a photosensitive molecule of the present invention may contain two or more photosensitive molecules for one molecule binding to a cancer-specific cell surface molecule. Such complex is expected to have higher antitumor activity than a complex containing only one photosensitive molecule, and reduce doses. In another embodiment, the complex of a binding molecule and a photosensitive molecule of the present invention may contain two or more molecules binding to a cancer-specific cell surface molecule for one photosensitive molecule. Such complex can target multiple types of cancers with different cancer-specific cell surface molecules, and can be used as a universal medicament with a wide range of applications.

### (1-1) Molecule Binding to Cancer-Specific Cell Surface Molecule

In the present invention, cancer-specific cell surface molecules are molecules expressed on the cell surface of cancer targeted by the medicament (I) of the present invention. Cancers targeted by the medicament (I) of the present invention are not particular restricted as long as they are cancers that express cancer-specific cell surface molecules or cancers that are accompanied by increased expression levels of genes encoding cancer-specific cell surface molecules. The “cancer” here is used synonymously with the terms “(malignant) tumor” and “(malignant) neoplasm”, and the target disease includes malignant diseases such as sarcoma, mesothelioma, reticuloendothelial system, lymphoid, and hematopoietic neoplastic disorders (e.g., myeloma, lymphoma, and leukemia). The medicament (I) of the present invention is administered intratumorally or intravenously. In a preferred embodiment, the target lesion is favorably a solid cancer, but the position of the cancer does not matter. Examples of the solid cancer includes head and neck cancer, oral cancer, thyroid cancer, esophageal cancer, stomach cancer, pancreatic cancer, colorectal cancer, lung cancer, breast cancer, liver cancer, bile duct cancer, skin cancer, bladder cancer, prostate cancer, uterine corpus cancer, cervical cancer, ovarian cancer, neuroblastoma, glioma, melanoma, sarcoma, mesothelioma, and the like.

In the present invention, the cancer-specific cell surface molecules are molecules specifically expressed in the above-mentioned cancers. Being specific here not only includes that the molecule is presented only on the surface of cell membrane of cancer, but also that the molecule is quantitatively large as compared with normal cells or tissues. The difference in quantitative factors may be, for example, about 2 to about 100 times. Examples of such cancer-specific cell surface molecule includes CD44, GPR87, EGFR, Mesothelin, Glypican-3, CEA, EpCAM, HER-2, CD133, integrin αvβ3, integrin αvβ6, laminin receptor, and the like.

In the present invention, the molecule binding to a cancer-specific cell surface molecule is not particularly limited as long as it is a molecule that binds to a molecule specifically expressed on the cell surface of the cancer targeted by the medicament (I) of the present invention. Examples of such binding molecules include antibody, binding peptide, nucleic acid aptamer, and the like, preferably antibody.

In the present invention, antibodies include both polyclonal antibodies and monoclonal antibodies. The antibody may include antibodies derived from any mammal, and may further belong to any immunoglobulin class of IgG, IgA, IgM, IgD, or IgE, preferably IgG. As the antibody, a commercially available antibody or an antibody stored at a research institution, each of which binds to the cancer-specific cell surface molecule of interest, may be used. Alternatively, those of ordinary skill in the art can produce antibodies according to conventionally known methods.

Antibodies include natural antibodies such as the aforementioned polyclonal antibodies and monoclonal antibodies (mAb), chimeric antibodies that can be produced using genetic recombination technique, humanized antibodies, and single-chain antibodies, as well as antibody fragments thereof. Antibody fragment refers to a partial region of the aforementioned antibody, and specifically includes Fab, Fab′, F(ab′)₂, SCAb, scFv, scFv-Fc, and the like.

In the present invention, binding peptides, unlike antibodies, are peptides that contain specific motif sequences that have binding activity to cancer-specific cell surface molecules. Examples of the motif sequence include RGD motif sequence (target: integrin αvβ3), YIGSR motif sequence (target: laminin receptor), and the like. The binding peptide may contain one or more motif sequences. Moreover, when two or more motif sequences are included, they may be the same motif sequences or mutually different motif sequences.

Antibodies or fragments thereof and binding peptides are indicated with the N-terminal (amino terminal) at the left end and the C-terminal (carboxyl terminal) at the right end, following the conventional peptide notation. The antibodies or fragments thereof and binding peptides used in the present invention may have any of a carboxyl group (—COOH), carboxylate (—COO⁻), amide (—CONH₂), and ester (—COOR) at the C-terminal.

As R in the ester, for example, a C₁₋₆ alkyl group such as methyl, ethyl, n-propyl, isopropyl, n-butyl, and the like; a C₃₋₈ cycloalkyl group such as cyclopentyl, cyclohexyl, and the like; a C₆₋₁₂ aryl group such as phenyl, α-naphthyl, and the like; a C₇₋₁₄ aralkyl group such as a phenyl-C₁₋₂ alkyl group such as benzyl, phenethyl, and the like; and an α-naphthyl-C₁₋₂ alkyl group such as α-naphthylmethyl and the like; a pivaloyloxymethyl group and the like are used.

Furthermore, the antibodies or fragments thereof and binding peptides used in the present invention also include those in which the amino group of the N-terminal amino acid residue is protected by a protecting group (e.g., C₁₋₆ acyl group such as C₁₋₆ alkanoyl such as formyl group, acetyl group, and the like), those in which the N-terminal glutamine residue that can be generated by cleavage in vivo became pyroglutamic acid, those in which substituents (e.g., —OH, —SH, amino group, imidazole group, indole group, guanidino group, and the like) on the side chain of amino acids in the molecule are protected with an appropriate protecting group (e.g., C₁₋₆ acyl group such as C₁₋₆ alkanoyl group such as formyl group, acetyl group, and the like), conjugated proteins such as so-called glycoprotein in which sugar chain is bound, and the like.

In the present invention, a nucleic acid aptamer refers to a nucleic acid that has binding activity to cancer-specific cell surface molecules. Nucleic acid aptamer can be RNA, DNA, modified nucleic acid, or a mixture thereof. Nucleic acid aptamer can also be in a linear or cyclic form.

When the nucleic acid aptamer is RNA, the sugar residues (e.g., ribose) of each nucleotide may be modified in order to improve stability and the like. Examples of the site to be modified in sugar residues include those in which the hydroxyl group at the 2′-position, 3′-position, and/or 4′-position of the sugar residue are/is replaced with another atom. Examples of the kind of modification include fluorination, alkoxylation, O-allylation, S-alkylation, S-allylation, and amination.

Furthermore, the sugar residue can also be BNA: Bridged nucleic acid (LNA: Linked nucleic acid), which forms a bridged structure at the 2′- and 4′-positions.

Nucleic acid aptamers can be produced using the SELEX method and an improved method thereof (e.g., Ellington et al., (1990), Nature, 346, 818-822; Tuerk et al., (1990), Science, 249, 505-510). In the SELEX method, nucleic acid aptamers that have stronger binding force to cancer-specific cell surface molecules are concentrated and selected by increasing the number of rounds or using competing substances. Therefore, nucleic acid aptamers with different binding forces, nucleic acid aptamers with different binding forms, and nucleic acid aptamers with the same binding force and binding form but different base sequences can be obtained by adjusting the number of rounds and/or changing the competition conditions in the SELEX method. In addition, the SELEX method includes an amplification process using PCR, and nucleic acid aptamers with more diverse sequences can be obtained by introducing mutations in the process, such as by using manganese ions.

The base length of the nucleic acid aptamer obtained by the SELEX method is about 80 nucleotides. It is preferred to shorten the length to a length permitting easy chemical synthesis (for example, chemical synthesis is possible with a length of about 60 nucleotides or less, more preferably about 50 nucleotides or less, further preferably 45 nucleotides or less).

### (1-2) Photosensitive Molecule

In the case of conventionally-known photosensitizers, the photosensitizer itself accumulates in tumors, and the energy difference between photoexcitation and photoemission of the photosensitizer acts on mitochondria, and the like, the resulting oxidative stress substances such as singlet oxygen and free radicals damage cells and lead them to apoptosis. On the other hand, the photosensitive molecule used in the present invention adheres to the target cancer via a molecule binding to a cancer-specific cell surface molecule and is excited by the light irradiated from the outside. The excited photosensitive molecules change from hydrophilic to hydrophobic and aggregate on the cell membrane together with binding molecules. As a result, the structure of the cell membrane is destroyed, and water molecules that enter the cell due to the difference in osmotic pressure between the inside and outside of the cell membrane cause the cell to rupture. This mechanism causes cell death (rupture of cancer cells) in an extremely short period of time, unlike apoptosis caused by photosensitizers. As such a photosensitive molecule, known molecules can be used. For example, tetrasodium salt of salotalocan (6-({[3-({(OC-6-13)-bis ({3-[bis (3-sulfopropyl) (3-sulfonatopropyl) azaniumyl]propyl}dimethylsilanolato-κO, κO′) [(phthalocyaninato (2-) κN²⁹, κN³⁰, κN³¹, κN³²)-1-yl]silicon}oxy)propoxy]carbonyl}amino) hexanoyl (C₇0H₉₆N₁₁O₂₄S₆Si₃; molecular weight: 1, 752.22) can be mentioned.

### (2) Adjuvant

The complex of a binding molecule and a photosensitive molecule of the present invention can release a large amount of cancer antigen into the surrounding area by rupturing cancer cells. This cancer antigen is taken up by dendritic cells and presented on MHC class I. Cancer antigens presented on dendritic cells can bind to cytotoxic T lymphocytes (CTLs) that express T cell receptors that can recognize them and transmit signals into CTLs. However, this alone does not sufficiently activate CTL. To sufficiently activate CTL, adjuvants need to activate dendritic cells via pattern recognition receptors. Therefore, the medicament (I) of the present invention contains an adjuvant (hereinafter referred to as the adjuvant of the present invention). Here, when the complex of a binding molecule and a photosensitive molecule of the present invention is delivered to cancer cells through blood vessels, it is considered that the complex initially accumulates most in cancer cells existing around the blood vessel. Cancer cells with accumulated complexes are induced to rupture in a short period of time by the light irradiated from the outside. As a result, a potential space is formed around the blood vessel, whereby the blood vessel is enlarged. It has been reported that this series of changes initially increases blood volume in the tumor tissue, along with which blood flow velocity reduces, tumor interstitial pressure lowers, and perfusion increases to cause nano-sized particles and drugs to leak into the remaining tumor tissue (Nanoscale. 2016 Jul. 7; 8 (25): 12504-12509). Therefore, the adjuvant of the present invention may be either nanoparticle or non-nanoparticle, but when the complex of a binding molecule and a photosensitive molecule of the present invention is administered intravenously, the adjuvant of the present invention is desirably nanoparticle. The particle size of the nanoparticles is not particularly limited as long as it is less than 1 μm, and is preferably 10 nm to 200 nm, more preferably 20 nm to 100 nm. The particle size can be measured using, for example, a particle size distribution measuring device such as Zetasizer Nano (Malvern) or the like. In the present specification, the “particle size” means an average particle size (number average) measured by a dynamic light scattering method.

The receptor for the adjuvant of the present invention is not particularly limited as long as it is a pattern recognition receptor expressed on innate immune cells such as dendritic cells and the like. Examples of the pattern recognition receptor include Toll-like receptor (TLR), NOD-like receptor (NLR), RIG-I like receptor (RLR), STING (stimulator of interferon genes), TYPE C lectin receptor (CLR), and the like. Among these, TLR excluding TLR3 or STING is preferred, and TLR excluding TLR3 is more preferred as the receptor for the adjuvant of the present invention. As TLR, TLR1, TLR2, TLR4, TLR5, TLR6, TLR7, TLR8, TLR9, TLR10, TLR11, and the like can be mentioned, among which TLR9 is preferred. On the other hand, since TLR3 does not activate CTL as a receptor for the adjuvant of the present invention, it is excluded from the receptors for the adjuvant of the present invention.

Examples of the ligand of the pattern recognition receptor include cytokines such as GM-CSF, IL-2, IL-7, IL-12, and the like, plant-derived components such as QS21 and the like, aluminum salts such as aluminum hydroxide, aluminum phosphate, aluminum chloride, and the like, oil-in-water emulsions (O/W emulsions) of squalene, mixture of squalene and tocopherol, and the like, water-in-oil emulsions (W/O emulsions) of Montanide ISA 51 and the like, synthetic polymer such as inulin, heme, and the like, lipid A such as 3-O-desacyl-4′-monophosphoryl lipid A (MPL) and the like, bacterium-derived proteins such as flagellin and the like, nucleic acids such as CpG ODN and the like, polysaccharides such as d-glucan, β-glucan, lentinan, and the like, complex of the aforementioned nucleic acid and the aforementioned polysaccharide, and the like. Preferably, polysaccharide and a complex of a nucleic acid and a polysaccharide can be mentioned.

From the above, the adjuvant of the present invention excludes ligands for TLR3. Furthermore, among the adjuvants other than TLR3 ligands, TLR9 ligand or STING ligand is preferred, and a ligand for TLR9 is more preferred.

Examples of the ligand for STING include nucleic acids such as c-di-GMP, cGAMP (e.g., 2′3′-cyclic guanosine monophosphate-adenosine monophosphate (2′3′-cGAMP)), and the like.

As the ligand for TLR9, a complex of a nucleic acid and a polysaccharide (hereinafter the complex of a nucleic acid and a polysaccharide of the present invention) is preferred.

The complex of a nucleic acid and a polysaccharide of the present invention is not limited as long as it can bind to TLR9 and activate dendritic cells. The complex preferably contains Type-K CpG oligodeoxynucleotide and polydeoxyadenylic acid (hereinafter the nucleic acid of the present invention) as the nucleic acid.

The CpG oligonucleotide (CpG ODN) contained in the nucleic acid of the present invention is not particularly limited as long as it is a short (about 20 base pairs), single-stranded synthetic DNA fragment containing an immunostimulatory CpG motif, activates dendritic cells (DCs) via TLR9, thereby activating cytotoxic T lymphocyte (CTL). As the CpG ODN to be used in the present invention, any of four types of type-K (also called type-B), TYPE-D (also called type-A), type-C, and type-P, respectively having different skeleton sequence and different immunostimulating property, can be used, and type-K is preferred.

The type-K CpG ODN contains multiple unmethylated CpG motifs with a non-palindromic structure. An unmethylated CpG motif is a short nucleotide sequence containing at least one cytosine (C)-guanine (G) sequence in which the 5-position of the cytosine in the cytosine-guanine sequence is not methylated.

The type-K CpG ODN contained in the nucleic acid of the present invention is preferably humanized. Being “humanized” means having agonist activity towards human TLR9. Therefore, the nucleic acid of the present invention containing humanized type-K CpG ODN has a dendritic cell activation function unique to humans.

The type-K CpG ODN preferably used in the present invention contains a nucleotide sequence having a length of 10 nucleotides or more and represented by the formula:

5′ N₁N₂N₃T-CpG-WN₄N₅N₆3′

wherein the central CpG motif is unmethylated, W is A or T, and N₁, N₂, N₃, N₄, N₅, and No may be any nucleotides.

In one embodiment, the type-K CpG ODN of the present invention is 10 nucleotides or more in length and contains a nucleotide sequence of the above formula. In the above-mentioned formula, the central 4-base CpG motif (T CpG W) only needs to be included in the 10 nucleotides, and does not necessarily need to be located between N₃ and N₄ in the above-mentioned formula. In the above-mentioned formula, N₁, N₂, N₃, N₄, N₅, and Ne may be any nucleotide, and the combination of at least one (preferably one) of Ni and N₂, N₂ and N₃, N₃ and N₄, N₄ and N₅, and N₅ and Ne may be a two-base CpG motif. When the aforementioned 4-base CpG motif is not located between N₃ and N₄, any two consecutive bases among the central 4 bases (4th to 7th bases) in the above-mentioned formula are CpG motifs, and other two bases may be any nucleotides.

The type-K CpG ODN more preferably used in the present invention contains a non-palindromic structure containing one or more CpG motifs. Furthermore, the type-K CpG ODN that is preferably used consists of a non-palindromic structure containing one or more CpG motifs.

Humanized type-K CpG ODN is generally characterized by a 4-base CpG motif consisting of TCGA or TCGT. One humanized type-K CpG ODN may contain only one, two or three or more of the 4-base CpG motif. In a preferred embodiment, the type-K CpG ODN contained in the nucleic acid of the present invention contains one, preferably two, and more preferably three or more 4-base CpG motifs consisting of TCGA or TCGT. When the type-K CpG ODN has two or more 4-base CpG motifs, these 4-base CpG motifs may be the same or different. They are not particularly limited as long as they have agonist activity against human TLR9.

The type-K CpG ODN contained in the nucleic acid of the present invention more preferably includes the nucleotide sequence shown in SEQ ID NO: 1.

The length of type-K CpG ODN is not particularly limited as long as the nucleic acid of the present invention can activate dendritic cells. It is generally 100 nucleotides or less (e.g., 10 to 75 nucleotides in length), preferably 50 nucleotides or less (e.g., 10 to 40 nucleotides in length), more preferably 30 nucleotides or less (e.g., 10 to 25 nucleotides in length), and most preferably 12 to 25 nucleotides in length.

The length of polydeoxyadenylic acid (dA) contained in the nucleic acid of the present invention is not particularly limited as long as it is long enough to form a triple helix structure together with a polysaccharide chain (preferably β-glucan). From the viewpoint of forming a stable triple helical structure, the length is generally 20 nucleotides or more, preferably 40 nucleotides or more, and more preferably 60 nucleotides or more. The length of poly dA is explained below in the case where the polysaccharide is β-glucan. Since a longer poly dA forms a more stable triple helix structure with β-glucan, no upper limit exists theoretically. However, when it is too long, variations occur in the length during synthesis of oligodeoxynucleotides. Therefore, it is generally 100 nucleotides or less, preferably 80 nucleotides or less. On the other hand, from the viewpoints of, in addition to forming the aforementioned stable triple helix structure, increasing the amount of the nucleic acid of the present invention bound per unit amount of β-glucan, avoiding variation in the length during synthesis of the nucleic acid, and improving the complex forming efficiency, the length of poly dA is preferably 20 to 60 nucleotides (specifically, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides), more preferably 30 to 50 nucleotides (30, 31, 32, 33, 34, 35, 36, 37, 38, 39, 40, 41, 42, 43, 44, 45, 46, 47, 48, 49, or 50 nucleotides), most preferably 30 to 45 nucleotides (30, 31, 32, 33, 34, 35, 36, 37, 38, 39, 40, 41, 42, 43, 44, or 45 nucleotides). In particular, when the length is 30 nucleotides or more, good complex forming efficiency is shown. The nucleic acid of the present invention has the activity of forming a triple helix structure with two β-glucans since it contains poly dA.

One molecule of the nucleic acid of the present invention may contain a plurality of type-K CpG ODNs and/or poly dA. Preferably, it contains one type-K CpG ODN and one poly dA, most preferably it consists of one type-K CpG ODN and one poly dA.

Exemplary CpG sequence includes, but is not limited to, K3 CpG (SEQ ID NO: 1: 5′-ATCGACTCTCGAGCGTTCTC-3′) and the like. In the nucleic acid of the present invention, the poly dA may be arranged on the 3′-side of the type-K CpG ODN, or the type-K CpG ODN may be arranged on the 3′-side of the poly dA. Preferably, it is characterized in that the poly dA is arranged on the 3′-side of the type-K CpG ODN. By this arrangement, the nucleic acid of the present invention may also enhance the anti-cancer action.

In the nucleic acid of the present invention, the type-K CpG ODN and poly dA may be directly linked by a covalent bond or may be linked via a spacer sequence. The spacer sequence refers to a nucleotide sequence that includes one or more nucleotides inserted between two adjacent constituent components. The length of the spacer sequence is not particularly limited as long as the nucleic acid of the present invention has the activity of activating dendritic cells. It is generally 1 to 10 nucleotides, preferably 1 to 5 nucleotides, more preferably 1 to 3 nucleotides. Most preferably, type-K CpG ODN and poly dA are directly linked by a covalent bond.

In addition to the type-K CpG ODN, poly dA, and an optional spacer sequence, the nucleic acid of the present invention may have additional nucleotide sequences at the 5′-terminal and/or the 3′-terminal. The length of the additional nucleotide sequence is not particularly limited as long as the nucleic acid of the present invention has the activity of activating dendritic cells. It is generally 1 to 10 nucleotides, preferably 1 to 5 nucleotides, more preferably 1 to 3 nucleotides.

In a preferred embodiment, the nucleic acid of the invention does not contain such additional nucleotide sequences at the 5′-terminal and/or the 3′-terminal. That is, the nucleic acid of the present invention preferably consists of type-K CpG ODN, poly dA, and an optional spacer sequence, and further preferably consists of type-K CpG ODN and poly dA.

In the most preferred embodiment, the nucleic acid of the present invention consists of type-K CpG ODN (specifically, for example, oligodeoxynucleotide consisting of the nucleotide sequence shown in SEQ ID NO: 1) and poly dA, and type-K CpG ODN is located at the 5′-terminal and the poly dA is located at the 3′-terminal of the oligodeoxynucleotide. Specifically, it is an oligodeoxynucleotide in which poly dA of generally 20 to 60 nucleotide length (preferably, 30 to 50 nucleotide length (30, 31, 32, 33, 34, 35, 36, 37, 38, 39, 40, 41, 42, 43, 44, 45, 46, 47, 48, 49, or 50 nucleotide length), more preferably 30 to 45 nucleotide length (30, 31, 32, 33, 34, 35, 36, 37, 38, 39, 40, 41, 42, 43, 44, or 45 nucleotide length), most preferably 40 nucleotide length) is bonded to the 3′-terminal of the oligodeoxynucleotide consisting of the nucleotide sequence shown in SEQ ID NO: 1.

The nucleic acid of the present invention may be appropriately modified to be resistant to in vivo degradation (e.g., degradation by exonuclease or endonuclease). Preferably, the modification includes phosphorothioate modification or phosphorodithioate modification. That is, some or all of the phosphodiester bonds in the nucleic acid of the present invention may be replaced with phosphorothioate bond or phosphorodithioate bond.

The nucleic acid of the present invention preferably includes modification with a phosphorothioate bond or a phosphorodithioate bond in the type-K CpG ODN, and more preferably, all of the phosphodiester bonds in the type-K CpG ODN are substituted with a phosphorothioate bond. In addition, the nucleic acid of the present invention preferably includes phosphorothioate bond or phosphorodithioate bond in the poly dA, and more preferably, all of the phosphodiester bonds in the poly dA are substituted with a phosphorothioate bond. Further preferably, all of the phosphodiester bonds in the oligodeoxynucleotide containing the humanized Type-K CpG oligodeoxynucleotide and polydeoxyadenylic acid of the present invention are substituted with a phosphorothioate bond. Most preferably, the nucleic acid of the present invention is a nucleic acid in which poly dA of generally 20 to 60 nucleotide length (preferably 30 to 50 nucleotide length (30, 31, 32, 33, 34, 35, 36, 37, 38, 39, 40, 41, 42, 43, 44, 45, 46, 47, 48, 49, or 50 nucleotide length), more preferably 30 to 45 nucleotide length (30, 31, 32, 33, 34, 35, 36, 37, 38, 39, 40, 41, 42, 43, 44, or 45 nucleotide length), most preferably 40 nucleotide length) is bonded to the 3′-terminal of humanized Type-K CpG oligodeoxynucleotide (e.g., SEQ ID NO: 1), and all of the phosphodiester bonds contained in the nucleic acid are substituted with a phosphorothioate bond. This is because the phosphorothioate bond is expected to achieve not only resistance to degradation, but also enhanced activity to activate dendritic cells and enhanced anti-cancer activity in the nucleic acid of the present invention. The phosphorothioate bond in the present specification has the same meaning as phosphorothioate skeleton, and the phosphodiester bond has the same meaning as phosphoric acid skeleton.

The nucleic acid of the present invention may be in any form of one-stranded, double-stranded, or triple-stranded form, but is preferably single-stranded.

The nucleic acid of the present invention is preferably isolated. “Isolation” means that an operation is performed to remove factors other than the target component and the naturally existing state no longer exists. The purity of the “isolated nucleic acid” (the percentage of the target nucleic acid weight in the total weight of the evaluation target) is generally 70% or more, preferably 80% or more, more preferably 90% or more, and further preferably 99% or more.

The nucleic acid of the present invention has superior activity of activating dendritic cells and is therefore useful as an adjuvant. Furthermore, the nucleic acid of the present invention has the property of forming a triple helix structure together with two polysaccharide chains (preferably β-glucan), and is therefore useful for preparing the adjuvant of the present invention.

While the aforementioned nucleic acid of the present invention contains K-type CpG ODN, it alone has poor activity to activate dendritic cells. Therefore, the nucleic acid of the present invention can compensate for the insufficient activity by forming a complex with a polysaccharide. The polysaccharide that forms a complex with the nucleic acid of the present invention includes d-glucan and β-glucan, and β-glucan is preferred.

Examples of the β-glucan used in the present invention include schizophyllan, scleroglucan, curdlan, pachyman, glyphoran, lentinan, laminaran, and the like, and schizophyllan is more preferred.

Schizophyllan (SPG) is a known soluble β-glucan derived from S. aeruginosa. SPG consists of a β-(1→3)-D-glucan main chain and one β-(1→6)-D-glucosyl side chain for three glucoses. SPG has been used as an intramuscular injection clinical drug for immune enhancement method against gynecological cancer for over 20 years, and its safety in vivo has been confirmed.

The complex of a nucleic acid and a polysaccharide of the present invention is formed by the association of a nucleic acid and a polysaccharide through a non-covalent bond such as electrostatic bond, Van der Waals bond, hydrogen bond, hydrophobicity interaction, or the like, or a covalent bond.

The complex of a nucleic acid and a polysaccharide of the present invention preferably has a triple helix structure. In a preferred embodiment, two of the three chains forming the triple helix structure are polysaccharide chains, and one is a polydeoxyadenylic acid chain in the nucleic acid of the present invention. The complex may partially include a portion that does not form a triple helical structure.

The composition ratio of the nucleic acid and polysaccharide in the complex of a nucleic acid and a polysaccharide of the present invention may vary depending on the chain length of polydeoxyadenylic acid in the nucleic acid, the length of the polysaccharide, and the like. For example, when the lengths of the polysaccharide chains and polydeoxyadenylic acid chains are equivalent, two polysaccharide chains and one nucleic acid of the present invention can associate to form a triple helix structure. Generally, since the chain length of polydeoxyadenylic acid is shorter than that of polysaccharide chains, multiple nucleic acids of the present invention are associated with two polysaccharide chains via polydeoxyadenylic acid and can form a triple helix structure.

The complex of a nucleic acid and a polysaccharide of the present invention is a complex containing humanized K-type CpG ODN and β-glucan (e.g., lentinan, schizophyllan, scleroglucan, curdlan, pachyman, glyphoran, laminaran), preferably a complex consisting of humanized type-K CpG ODN and β-glucan (e.g., schizophyllan). More preferably, it is a complex (e.g., K3-dA20-60-SPG) consisting of an oligodeoxynucleotide consisting of the nucleotide sequence shown in SEQ ID NO: 1, in which a polydeoxyadenylic acid of 20 to 60 nucleotide length (specifically, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotide length) is bonded to the 3′-side of the oligodeoxynucleotide, and all of the phosphodiester bonds are substituted with a phosphorothioate bond, and β-glucan (e.g., schizophyllan), further preferably, a complex (e.g., K3-dA30-50-SPG) consisting of the nucleotide sequence shown in SEQ ID NO: 1, in which a polydeoxyadenylic acid of 30 to 50 nucleotide length (specifically, 30, 31, 32, 33, 34, 35, 36, 37, 38, 39, 40, 41, 42, 43, 44, 45, 46, 47, 48, 49, or 50 nucleotide length) is bonded to the 3′-side of the oligodeoxynucleotide, and all of the phosphodiester bonds are substituted with a phosphorothioate bond, and β-glucan (e.g., schizophyllan), most preferably, a complex (K3-dA30-45-SPG) consisting of the nucleotide sequence shown in SEQ ID NO: 1, in which a polydeoxyadenylic acid of 30 to 45 nucleotide length (specifically, 30, 31, 32, 33, 34, 35, 36, 37, 38, 39, 40, 41, 42, 43, 44, or 45 nucleotide length) is bonded to the 3′-side of the oligodeoxynucleotide, and all of the phosphodiester bonds are substituted with a phosphorothioate bond, and β-glucan (e.g., schizophyllan).

The method for preparing the complex of a nucleic acid and a polysaccharide of the present invention can be performed using known means, for example, the conditions similar to those described in JP-A-2008-100919. That is, β-glucan, which naturally exists as a triple helix structure, is dissolved in a non-protonic organic polar solvent (dimethyl sulfoxide (DMSO), acetonitrile, acetone, etc.) or an alkali aqueous solution (sodium hydroxide, potassium hydroxide, ammonia, calcium hydroxide, etc.) to give single chain β-glucan. A solution of the thus-obtained single chain β-glucan and a solution of the nucleic acid of the present invention (an aqueous solution, a buffered aqueous solution with a pH around neutrality, or an acidic buffered aqueous solution, preferably an aqueous solution or a buffered aqueous solution with a pH around neutrality) are mixed, the pH is adjusted again to around neutrality if necessary, and the mixture is retained for an appropriate period of time, for example, at 5° C. overnight. As a result, the two β-glucan chains and the poly dA chain in the nucleic acid form a triple helix structure, thereby forming the complex of a nucleic acid and a polysaccharide of the present invention. By subjecting the generated complex to purification by size-exclusion chromatography, ultrafiltration, dialysis, and the like, oligodeoxynucleotides not forming a complex can be removed. In addition, by purifying the generated complex by anion exchange chromatography, β-glucan not forming a complex can be removed. The complex can be appropriately purified by the above-mentioned methods.

Formation of the complex of a nucleic acid and a polysaccharide of the present invention can be confirmed by, but not limited to, for example, measuring conformation change by CD (circular polarization dichroism) spectrum, UV absorption shift by size-exclusion chromatography, gel electrophoresis, microchip electrophoresis, or capillary electrophoresis.

The mixing ratio of the nucleic acid of the present invention and the polysaccharide can be appropriately determined in consideration of the length of the poly dA chain, and the like, but the molar ratio (SPG/ODN) is generally 0.02 to 2.0, preferably 0.1 to 0.5.

In one embodiment, the complex of a nucleic acid and a polysaccharide of the present invention takes the form of rod-shaped particles. The particle size is equivalent to the particle size of a particle formed using β-glucan (e.g., schizophyllan) which is natural and has a triple helix structure as a material, and the average particle size is generally 10 to 100 nm, preferably 20 to 50 nm. The particle size can be measured by dissolving the complex in water and by a dynamic light scattering method at 80° C. using a Malvern Instruments Zeta Sizer.

The complex of a nucleic acid and a polysaccharide of the present invention is preferably isolated. The purity of the “isolated complex” (the percentage of the target complex weight in the total weight of the evaluation target) is generally 70% or more, preferably 80% or more, more preferably 90% or more, and further preferably 99% or more.

### 2. Administration of Medicament (I)

The complex of a binding molecule and a photosensitive molecule of the present invention obtained as described above and the adjuvant of the present invention can be provided as a medicament. In the medicament (I) of the present invention, since both the complex of a binding molecule and a 2.5 photosensitive molecule of the present invention as an active ingredient and the adjuvant of the present invention are of low toxicity, they can be administered as a liquid as it is, or as an appropriate dosage form of a pharmaceutical composition, to mammals that have developed cancers, orally or parenterally (e.g., intratumoral administration, intravascular administration, and the like). Parenteral administration is preferred, and intratumoral administration is more preferred. Examples of the mammal to be the subject of administration include rodents such mouse and the like, pets such dog and the like, livestocks such as swine, horse, and the like, primates such as human, monkey, orangutan, chimpanzee, and the like, and the like, and human is particularly preferred.

As pharmaceutical compositions for parenteral administration, for example, injection, suppository, and the like are used, and injections may include dosage forms such as intratumoral injection, intravenous injection, intramuscular injection, drip injection, and the like. Such injections can be prepared according to known methods. As a method for preparing an injection, for example, injections can be prepared by dissolving, suspending, or emulsifying the above-mentioned complex of a binding molecule and a photosensitive molecule of the present invention and the adjuvant of the present invention in a sterile aqueous solution or oily liquid generally used for injections. Examples of aqueous solution for injections include physiological saline, isotonic solutions containing glucose and other auxiliary agents, and it may be used in combination with suitable solubilizing agents such as alcohol (e.g., ethanol), polyalcohol (e.g., propylene glycol, polyethylene glycol), nonionic surfactant [e.g., polysorbate 80, HCO-50 (polyoxyethylene (50 mol) adduct of hydrogenated castor oil)], and the like. As the oily liquid, for example, sesame oil, soybean oil, and the like are used, and benzyl benzoate, benzyl alcohol, and the like may be used in combination as a dissolution aid. The prepared injection solution is preferably filled into suitable ampoules. Suppositories used for rectal administration may be prepared by mixing the above-mentioned complex of a binding molecule and a photosensitive molecule of the present invention and the adjuvant of the present invention with a general suppository base.

As a pharmaceutical composition for oral administration, a solid or liquid dosage form, specifically tablet (including sugar-coated tablet, film-coated tablet), pill, granule, powder, capsule (including soft capsule), syrup, emulsion, suspension, and the like can be mentioned. Such compositions may be produced by known methods and may contain carriers, diluents, or excipients generally used in the field of preparation formulation. As carriers and excipients for tablets, for example, lactose, starch, sucrose, and magnesium stearate are used.

When the medicament (I) of the present invention is administered to an adult cancer patient, it can be administered, for example, directly to the tumor site or surrounding area, or intravascularly (e.g., intravenously). The dose thereof can be appropriately determined by a doctor or medical professional, taking into consideration the size of the tumor, age, body weight, condition, and the like of the patient. When the complex of a binding molecule and a photosensitive molecule of the present invention is a complex of an antibody and IR700, the dose of the complex can be, for example, 300 mg to 1, 200 mg per m² of body surface area by intravenous administration. For intratumoral administration, the dose can be 30 mg to 120 mg, depending on the tumor size. In addition, when the adjuvant of the present invention is K3-SPG, the dose of K3-SPG can be, for example, 0.001, 1, 5, 10, 15, 100, or 1,000 mg/kg body weight per dose. The number of administrations can also be appropriately determined by a doctor or medical professional, taking into consideration the size of the tumor, age, weight, condition, and the like of the patient.

The medicament (I) of the present invention may be prepared as a combination drug containing the complex of a binding molecule and a photosensitive molecule of the present invention and the adjuvant of the present invention, or it may also be prepared as a kit containing each prepared as a separate agent in one package. Furthermore, the medicament (Ia) of the present invention may be prepared as a kit provided in one package as an agent of the complex of a binding molecule and a photosensitive molecule of the present invention, and as a kit for use in combination with the adjuvant of the present invention. In addition, the medicament (Ib) of the present invention may be prepared as a kit provided in one package as an agent of the adjuvant of the present invention, and as a kit for use in combination with the complex of a binding molecule and a photosensitive molecule of the present invention. When prepared as a kit for combined use, the usage and dosage in the package insert states that one embodiment of the usage and dosage may be combined use of the complex of a binding molecule and a photosensitive molecule of the present invention or the adjuvant of the present invention.

When the medicament (I), (Ia), or (Ib) of the present invention is each an additive containing the complex of a binding molecule and a photosensitive molecule of the present invention and the adjuvant of the present invention, the complex of a binding molecule and a photosensitive molecule of the present invention and the adjuvant of the present invention are administered by simultaneous administration by the same administration route. Examples of the administration route for the additive include intratumoral administration, intravenously administration, and the like.

When the medicament (I) of the present invention is a kit separately containing the complex of a binding molecule and a photosensitive molecule of the present invention, and the adjuvant of the present invention, the complex of a binding molecule and a photosensitive molecule of the present invention and the adjuvant of the present invention may be subjected to any of simultaneous administration by the same administration route, administration in a staggered manner by the same administration route, simultaneous administration by different administration routes, and administration in a staggered manner by different administration routes. Examples of the administration route for the kit include intratumoral administration, intravenous administration, and the like. Therefore, in some embodiments, the complex of a binding molecule and a photosensitive molecule of the present invention may be intratumorally administered and the adjuvant of the present invention may be intratumorally administered. In another embodiment, the complex of a binding molecule and a photosensitive molecule of the present invention may be intravenously administered and the adjuvant of the present invention may be intratumorally administered. In still another embodiment, the complex of a binding molecule and a photosensitive molecule of the present invention may be intravenously administered and the adjuvant of the present invention may be intravenously administered. In still another embodiment, the complex of a binding molecule and a photosensitive molecule of the present invention may be intratumorally administered and the adjuvant of the present invention may be intravenously administered.

After the medicament (I) of the present invention is administered, near-infrared ray is irradiated to the cancer lesion. The near-infrared rays are irradiated generally for 6 hr to 72 hr, preferably 12 hr to 60 hr, more preferably 18 hr to 48 hr, further preferably 24 hr to 36 hr, after administration of the medicament (I) of the present invention. The wavelength of the near-infrared rays to be irradiated is not limited as long as it is a wavelength suitable for exciting photosensitive molecules, and is generally about 685 nm to about 695 nm. The amount of far infrared rays to be irradiated can be appropriately determined by a doctor or medical professional, and can be, for example, 10 J/cm² to 200 J/cm².

### 3. Administration of Medicament (II)

The present invention also provides a medicament for cancer treatment, which includes a complex of a photosensitive molecule and a molecule binding to a cancer-specific cell surface molecule, and nanoparticles (hereinafter referred to as medicament (II) of the present invention). The medicament (II) of the present invention may be a medicament for cancer treatment, containing a complex of a photosensitive molecule and a molecule binding to a cancer-specific cell surface molecule, which can be used in combination with nanoparticles. In addition, the medicament (II) of the present invention may be a medicament for cancer treatment, containing nanoparticles, which can be used in combination with a complex of a photosensitive molecule and a molecule binding to a cancer-specific cell surface molecule. The nanoparticles are, for example, nanoparticles having immunostimulatory activity or nanoparticles having anti-cancer activity. The nanoparticles having immunostimulatory activity include nanoparticulated adjuvants. In other words, the aforementioned adjuvant of the present invention only needs to be nanoparticulated and, for example, a ligand for TLR9 is preferred, and as such ligand, a complex of a nucleic acid and a polysaccharide is preferred. Examples of the nanoparticle having anti-cancer activity include nanoparticulated anti-cancer agents. Examples of the anti-cancer agent include, but are not limited to, paclitaxel, cetuximab, Gefitinib, cyclophosphamide, imatinib, cisplatin, afacinib, gemcitabine, lapatinib, erlotinib, docetaxel, Opdivo, taxol, veltuzumab, trastuzumab, panitumumab, rituximab, and the like.

After the medicament (II) of the present invention is administered, near-infrared ray is irradiated to the cancer lesion, as in the case of the medicament (I) of the present invention. The timing of irradiating near-infrared ray, the wavelength of irradiated near-infrared ray, and the irradiation amount of far-infrared ray may be the same as those in the case of the medicament (I) of the present invention.

The present invention is explained below with reference to Examples. However, the present invention is not limited to these examples.

### EXAMPLE

### Cell Line

As a pancreatic cancer model cell line, KPC-N cells established from genetically modified pancreatic cancer model mice (KrasLSL-G12D/+, Trp53LSL-R172H/+, Pdx1-Cre: KPC mice) were used. In addition, MC38 cells were used as a cell line for a colorectal cancer model.

### Photoimmunotherapy

InVivoMAb-anti-mouse/human CD44 (clone IM7: BioXcell) was used as the antibody for photoimmunotherapy. In addition, IRDye 700DX NHS (LI-COR) was used as a fluorescent dye for photoimmunotherapy. IR700-conjugated CD44 antibody (anti-CD44 Ab-IR700) was prepared using IRDye 700DX NHS and InVivoMAb-anti-mouse/human CD44 according to a previous report (Mitsunaga et al. Nat Med. 17, 2011), and used as a substance for photoimmunotherapy. Furthermore, MLL-III-690-1300 mW-3%-LED (CNI) was used as an infrared irradiation device to excite the fluorescent dye.

### Adjuvant

### (1) TLR9 Ligand

K3-SPG or Alexa647-K3-SPG, which was created in the laboratory of Professor Ken Ishii of the Institute of Medical Science, the University of Tokyo, was used. K3-SPG is a TLR9 ligand and is a complex of a nucleic acid and a polysaccharide. This K3-SPG is a nanoparticulated adjuvant. Alexa647-K3-SPG is Alexa647-labeled K3-SPG. K3-SPG and Alexa647-K3-SPG were synthesized, for example, by the method described in WO 2015/041318. That is, a CpG ODN in which poly dA is provided on the 3′-side of type-K CpG ODN (SEQ ID NO: 2:5′-ATCGACTCTCGAGCGTTCTC-40 mer A-3′) was synthesized, and a complex of the synthesized CpG ODN and SPG was formed.

More specifically, the CpG ODN of the Example is shown in Table 1 and was synthesized by GeneDesign, Inc. (s in the sequence of Table 1 indicates that the phosphodiester bond between nucleosides is replaced with a phosphorothioate bond).

TABLE 1

| adjuvant | 5′-base sequence-3′ |
|---|---|
| CpG ODN | AsTsCsGsAsCsTsCsTsCsGsAsGsCsGsTsTsCs |
| | TsCsAsAsAsAsAsAsAsAsAsAsAsAs |
| | (SEQ ID NO: 2) |

This oligodeoxynucleotide was synthesized using the conventional solid-phase phosphoramidite method (Nucleic Acids in Chemistry and Biology, 3. Chemical synthesis (1990) ed. G. Michael Blackburn and Michael J. Gait. Oxford University Press).

A complex of CpG ODN and SPG was formed by the following procedure. 7.22 mg of K3-dA40 was dissolved in water (3.7 mL). 15 mg of SPG was dissolved in 0.25 N NaOH (1 mL). The SPG solution was added to DNA and mixed well, 1 mL of 330 mM NaH₂PO₄ was added to the DNA/SPG solution, and the mixture was maintained overnight at 4° C., whereby formation of a complex was completed. The molar ratio (MSPG/MDNA) was fixed at 0.27. Complex formation was confirmed using a microchip electrophoresis device (SHIMADZU: MultiNA).

### (2) Sting

2′ 3′-CGAMP (2′ 3′-cGAMP VacciGrade™: InvivoGen), which is a STING ligand, was used.

### (3) TLR3 Ligand

PolyI:C (Poly (I:C) (HMW) VacciGrade™: InvivoGen), which is a TLR3 ligand, was used.

### Example 1 Photoimmunotherapy/Adjuvant Combination Therapy Using Pancreatic Cancer Model (1)

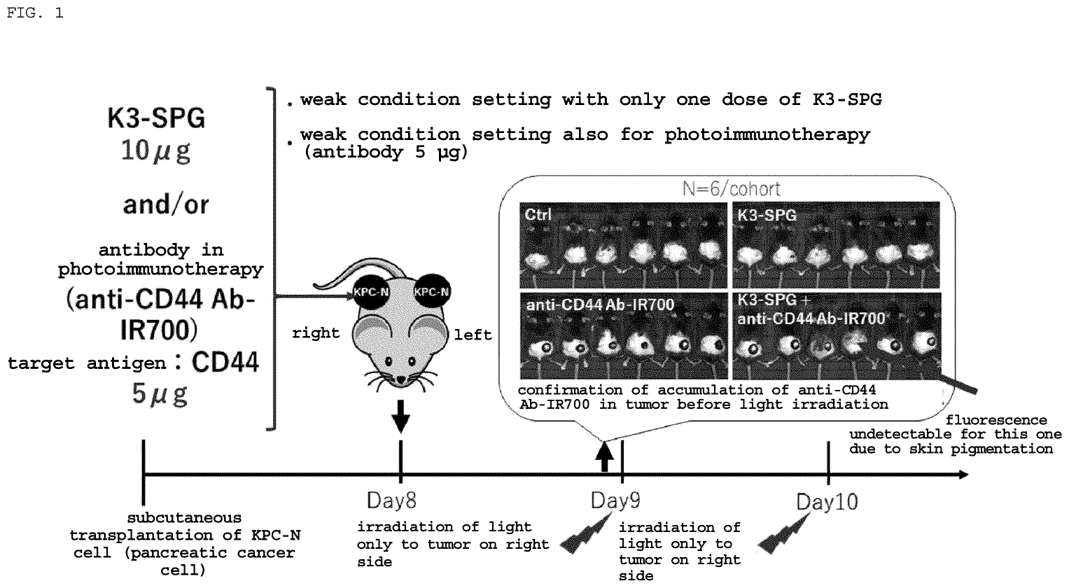

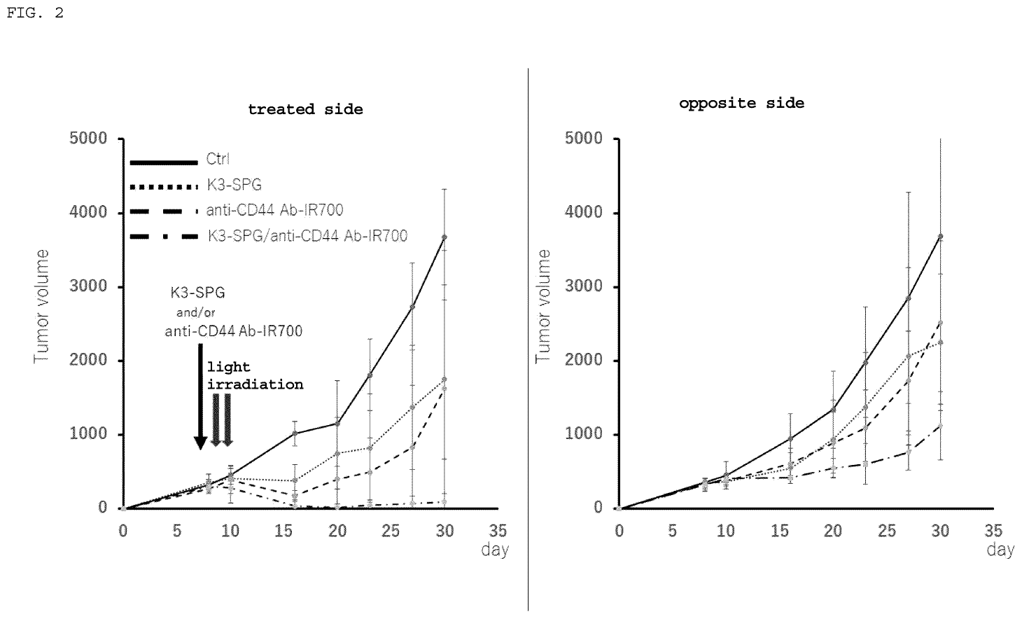

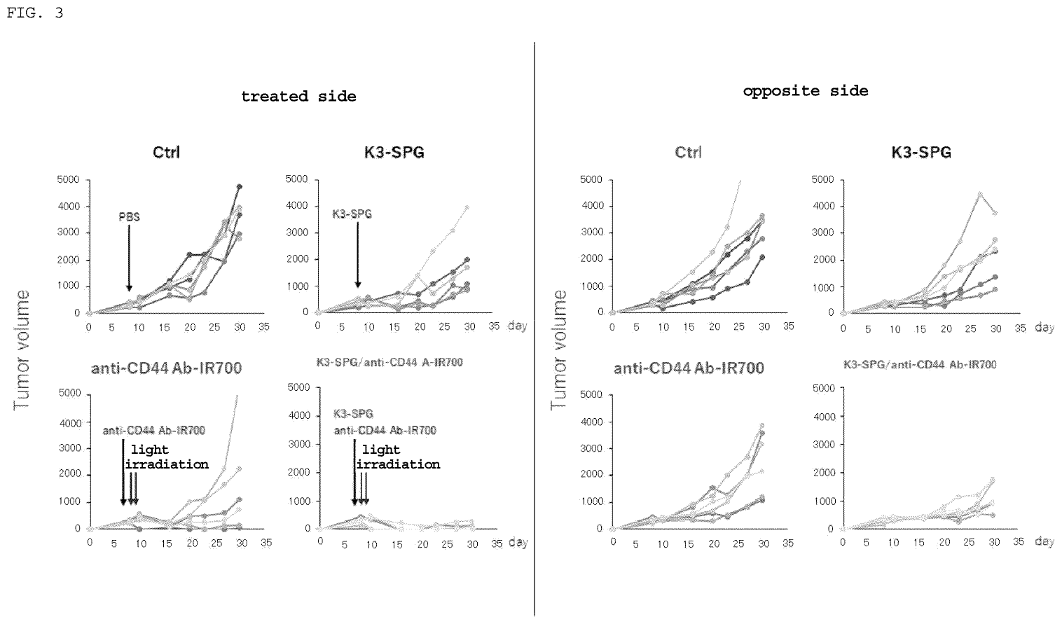

1.5×10⁶ KPC-N cells were subcutaneously transplanted into one left area and one right area on the back of 6- to 8-week-old C57BL/6 mice, and 4 groups (each group N=6) of non-treatment group (Ctrl), adjuvant administration group (K3-SPG), photoimmunotherapy group (anti-CD44 Ab-IR700), and combination therapy group (K3-SPG/anti-CD44 Ab-IR700) were established. To the tumor on the right side of the back of the mice on day 8 after cell transplantation were topically injected (intratumoral administration) 100 μl of PBS for the non-treatment group, 10 μg of K3-SPG dissolved in 100 μl of PBS for the adjuvant administration group, 5 μg of anti-CD44 Ab-IR700 dissolved in 100 μl of PBS for the photoimmunotherapy group, and 10 μg of K3-SPG dissolved in 100 μl of PBS and 5 μg of anti-CD44 Ab-IR700 for the combination therapy group. On days 9 and 10 after transplantation, near-infrared ray (wavelength: 690 nm) was irradiated at 100 J/cm² to the tumor on the right side of the back (FIG. 1). The size of the left and right tumors (calculated by major axis×minor axis×minor axis×½) was monitored over time (FIGS. 2, 3).

As a result, the tumor on the right side of the back of the mice continued to grow in the non-treatment group. In the adjuvant administration group and the photoimmunotherapy group, the tumor on the right side of the back once shrank, but continued to grow slowly thereafter. However, in the combination therapy group, the tumor on the right side of the back continued to shrink immediately after administration, and eventually almost disappeared. In addition, the tumor on the left side of the back of the mice continued to grow all the time in the non-treatment group, similar to the tumor on the right side of the back. In the adjuvant administration group and the photoimmunotherapy group, the tumor on the left side of the back continued to grow, although at a slower rate than in the non-treatment group. On the other hand, in the combination therapy group, the tumor on the left side of the back continued to grow, but at a far slower rate than in the adjuvant administration group and the photoimmunotherapy group.

It is considered that the disappearance of the tumor on the right side of the back in the combination therapy group was realized by the direct destruction of the tumor by photoimmunotherapy, followed by the attack on the remaining tumor by anti-tumor immunity strongly induced by the antigen and adjuvant released from the destroyed tumor. In addition, it is appreciated that the antitumor immunity was more strongly induced in the combination therapy group than in the adjuvant administration group and the photoimmunotherapy group since the growth rate of the tumor on the left side of the back in the combination therapy group was slower than in the adjuvant administration group and the photoimmunotherapy group.

Existing PIT aims to selectively destroy cancer cells, and does not intend to induce cancer immunity. In other words, PIT does not directly amplify LIIA, but relies on normal reactions within the body. Accordingly, PIT has been considered to not afford a sufficient effect as an in situ vaccine. On the other hand, the photoimmunotherapy/adjuvant combination therapy of Example 1 directly amplifies LIIA and can be expected to show a superior LTAR effect. That is, the photoimmunotherapy/adjuvant combination therapy of Example 1 is an immunotherapy (cancer vaccine therapy) based on a new concept of activating cancer immunity with an action mechanism completely different from that of checkpoint inhibitors. Therefore, even though it is a therapeutic intervention topically on the tumor, side effects can be suppressed while inducing systemic antitumor effects.

Patent Literature 1 (JP 2018-528268 A) describes that when FaDu cells treated with cetuximab IRDye 700DX were irradiated with light and co-cultured with human dendritic cells, and the collected human dendritic cells were treated with polyI:C, increased CD80 and CD86 expression levels were achieved as compared with the control without light irradiation. However, it does not show an antitumor effect. In addition, poly I: C is not a TLR9 ligand and is not a complex of a nucleic acid and a polysaccharide. Therefore, the content described in Patent Literature 1 is different from the photoimmunotherapy/adjuvant combination therapy of Example 1.

### Example 2 Photoimmunotherapy/Adjuvant Combination Therapy Using Colorectal Cancer Model

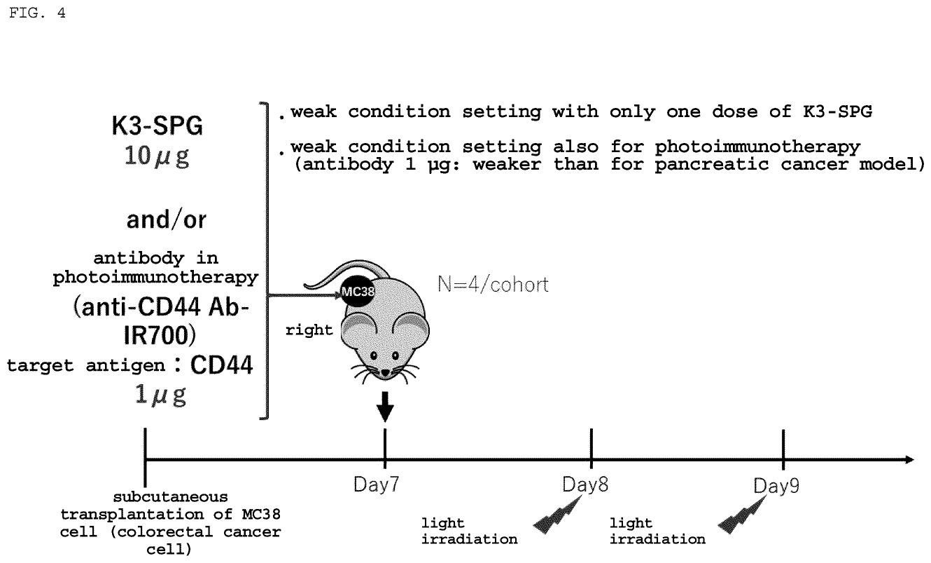

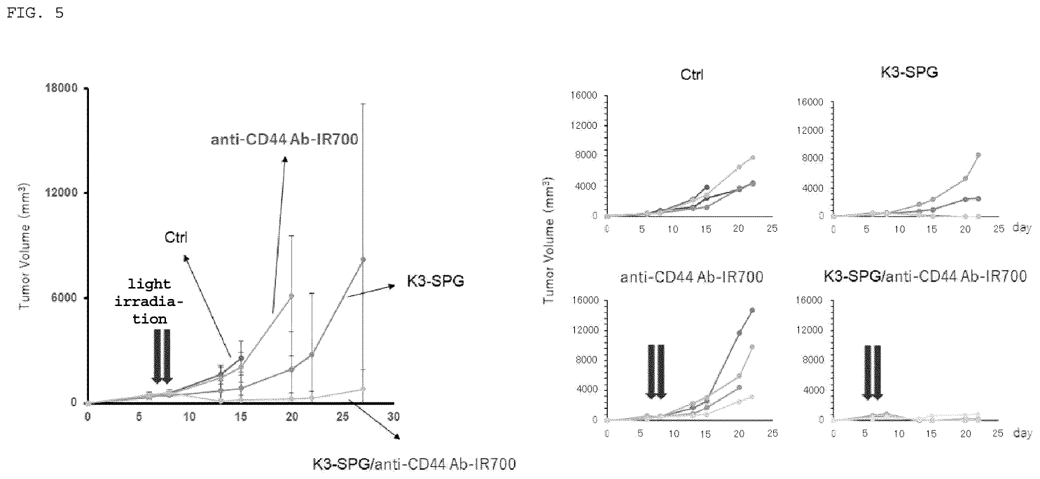

1.5×10⁶ MC38 cells were subcutaneously transplanted into the back (one right side) of 6- to 8-week-old C57BL/6 mice, and 4 groups (each group N=4) of non-treatment group (Ctrl), adjuvant administration group (K3-SPG), photoimmunotherapy group (anti-CD44 Ab-IR700), and combination therapy group (K3-SPG/anti-CD44 Ab-IR700) were established. To the tumor on the right side of the back of the mice on day 7 after cell transplantation were topically injected (intratumoral administration) 100 μl of PBS for the non-treatment group, 10 μg of K3-SPG dissolved in 100 μl of PBS for the adjuvant administration group, 1 μg of anti-CD44 Ab-IR700 dissolved in 100 μl of PBS for the photoimmunotherapy group, and 10 μg of K3-SPG dissolved in 100 μl of PBS and 1 μg of anti-CD44 Ab-IR700 for the combination therapy group. On days 8 and 9 after transplantation, near-infrared ray (wavelength: 690 nm) was irradiated at 100 J/cm² to the tumor on the right side of the back (FIG. 4). The size of the tumor (calculated by major axis×minor axis×minor axis×½) was monitored over time (FIG. 5).

As a result, the tumor on the right side of the back of the mice continued to grow in the non-treatment group. In the adjuvant administration group and the photoimmunotherapy group, the tumor on the right side of the back continued to grow, although at a slower rate than in the non-treatment group. However, in the combination therapy group, the tumor on the right side of the back continued to shrink immediately after administration, and eventually almost disappeared.

Similar to the pancreatic cancer model of Example 1, it is considered that the disappearance of the tumor on the right side of the back in the combination therapy group was realized by the direct destruction of the tumor by photoimmunotherapy, followed by the further attack on the tumor by anti-tumor immunity strongly induced by the antigen and adjuvant released from the tumor. That is, the photoimmunotherapy/adjuvant combination therapy of Example 2 is an immunotherapy (cancer vaccine therapy) based on a new concept of activating cancer immunity with an action mechanism completely different from that of checkpoint inhibitors. Thus, even though it is a therapeutic intervention topically on the tumor, side effects can be suppressed while inducing systemic antitumor effects.

### Example 3 Photoimmunotherapy/Adjuvant Combination Therapy Using Pancreatic Cancer Model (2)

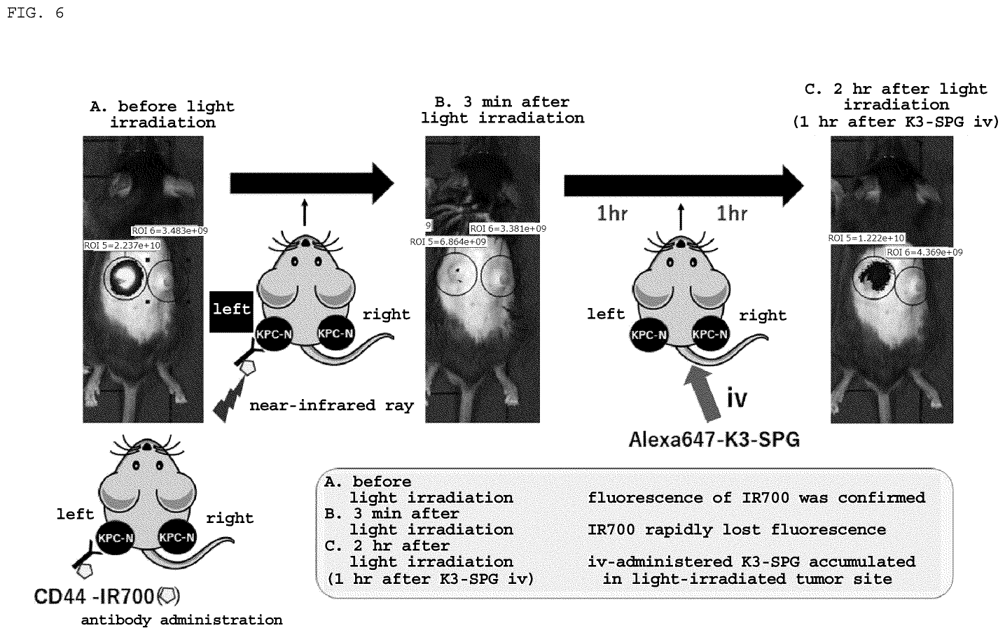

1.5×10⁶ KPC-N cells were subcutaneously transplanted into one left area and one right area on the back of 6- to 8-week-old C57BL/6 mice. To the tumor on the left side of the back of the mice on day 7 after cell transplantation was topically injected (intratumoral administration) 5 μg of anti-CD44 Ab-IR700 dissolved in 100 μl of PBS. On day 8 after transplantation, the intratumoral IR700 fluorescence signal was monitored using the IVIS system. Then, near-infrared ray (wavelength: 690 nm) was irradiated to the left side tumor at 100 J/cm². After 3 minutes, the IR700 fluorescence signal within the tumor was monitored using the IVIS system (the IR700 fluorescence signal disappeared due to near-infrared irradiation). One hour later, Alexa647-K3-SPG (10 μg) dissolved in 100 μl of PBS was administered intravenously (iv) from the penile vein. Furthermore, one hour later, the fluorescence signal of Alexa647 was monitored using the IVIS system. As a result, intravenously administered Alexa647-K3-SPG was strongly accumulated in tumors treated with photoimmunotherapy, and hardly accumulated in tumors not treated with photoimmunotherapy (FIG. 6). That is, Alexa647-K3-SPG was selectively accumulated in tumors treated with photoimmunotherapy.

### Example 4 Photoimmunotherapy/Adjuvant Combination Therapy Using Pancreatic Cancer Model (3)

1.5×10⁶ KPC-N cells were subcutaneously transplanted to the back of 6- to 8-week-old C57BL/6 mice, and 4 groups (each group N=6) of non-treatment group (Control), adjuvant administration group (2′3′-cGAMP), photoimmunotherapy group (anti-CD44 Ab-IR700), and combination therapy group (2′3′-CGAMP/anti-CD44 Ab-IR700) were established. To the tumor of the mice on day 6 after cell transplantation were topically injected (intratumoral administration) 5 μg of 2′3′-cGAMP for the adjuvant administration group, 5 μg of anti-CD44 Ab-IR700 for the photoimmunotherapy group, 5 μg of 2′3′-CGAMP and 5 μg of anti-CD44 Ab-IR700 for the combination therapy group. On days 7 and 8 after transplantation, near-infrared ray (wavelength: 690 nm) was irradiated at 100 J/cm². The size of the tumor (calculated by major axis×minor axis×minor axis×½) was monitored over time.

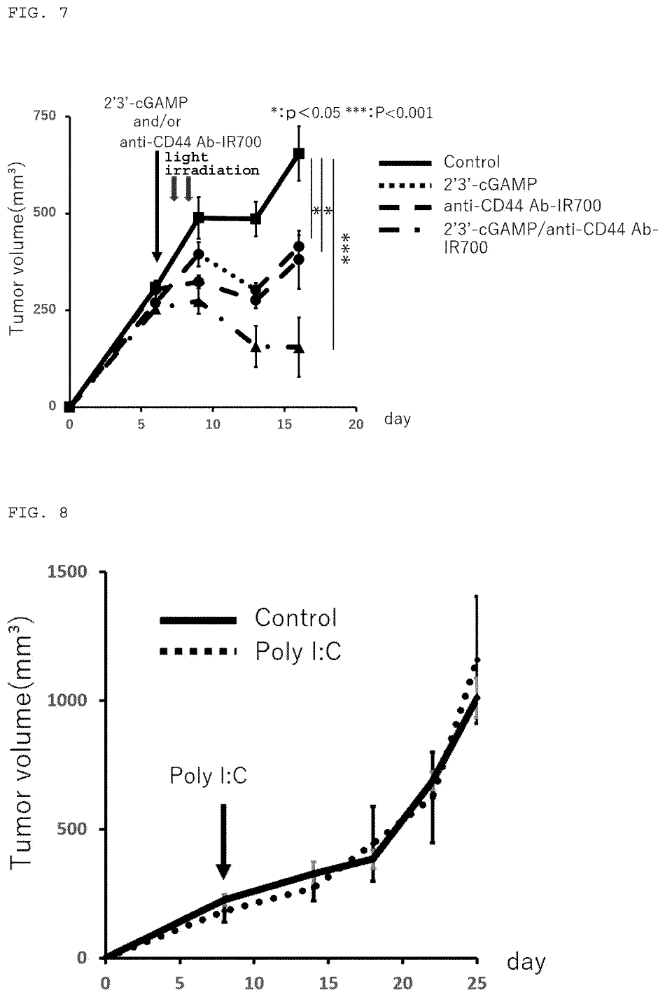

As a result, an enhanced antitumor effect was confirmed in the combination therapy group as compared with the adjuvant administration group and the photoimmunotherapy group (FIG. 7).

### Example 5 Antitumor Effect by TLR3 Ligand

1.5×10⁶ KPC-N cells were subcutaneously transplanted to the back of 6- to 8-week-old C57BL/6 mice, and two groups (each group N=6) of non-treatment group (Control) and polyI:C group were established. To the tumor of the mice on day 8 after subcutaneous transplantation was topically injected (intratumoral administration) 50 μg of polyI:C. The size of the tumor (calculated by major axis×minor axis×minor axis×½) was monitored over time to find no antitumor effect by polyI:C was found (FIG. 8).

### Example 6 Induction of Immunological Memory by Adjuvant Administration/Photoimmunotherapy Combination Therapy

1.5×10⁶ KPC-N cells were subcutaneously transplanted to the back of 6- to 8-week-old C57BL/6 mice, and 2 groups (each group N=6) of non-treatment group (Control) and adjuvant administration/photoimmunotherapy combination therapy group (K3-SPG/anti-CD44 Ab-IR700) were established. To the tumor of the mice on day 8 after cell transplantation were intratumorally administered 100 μl of PBS for the non-treatment group, and 10 μg of K3-SPG dissolved in 100 μl of PBS and 5 μg of IR700-conjugated anti-CD44 antibody (Ab-IR700) for the combination therapy group. On days 9 and 10 after transplantation, near-infrared ray (wavelength: 690 nm) was irradiated to the subcutaneous tumor at 100 J/cm². The size of the tumor (calculated by major axis×minor axis×minor axis×½) was monitored over time.

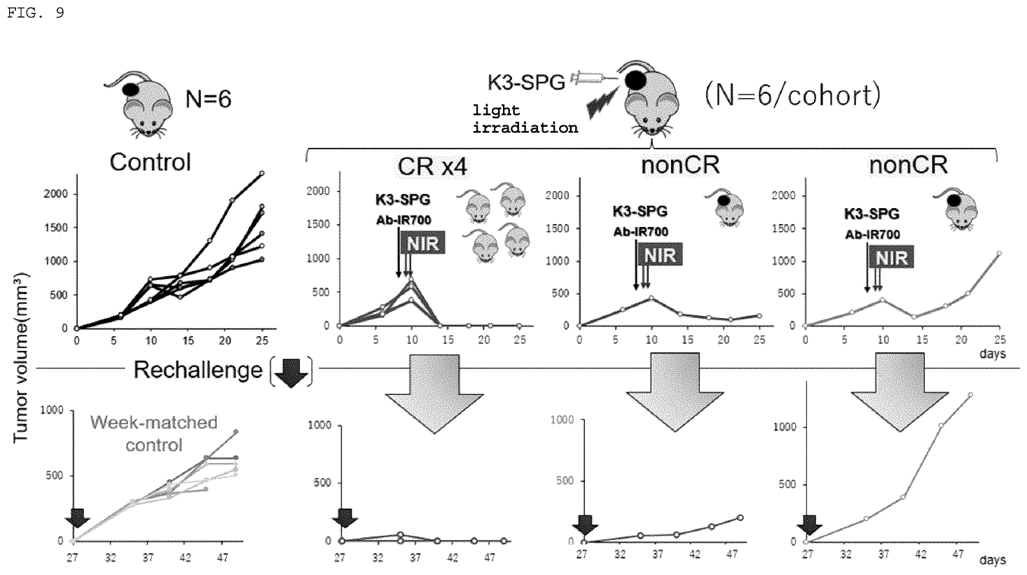

As a result, the tumor continued to grow in the non-treatment group, but in the combination therapy group, the tumor completely disappeared in 4 out of 6 mice (CR mice) and the tumor remained in 2 mice (nonCR mice) (FIG. 9).

Furthermore, on day 27 after transplantation, 1.5×10⁶ KPC-N cells were transplanted again to each of the above-mentioned four CR mice, two nonCR mice, and six week age-matched control mice.

As a result, proliferation of the re-transplanted cells was observed in the control mice, but no engraftment of the re-transplanted cells was observed in the four CR mice (FIG. 9). In addition, proliferation of re-transplanted cells was observed in two nonCR mice. These results suggest that immunological memory was induced in the CR mice that showed a strong antitumor effect in the combination treatment group.

### INDUSTRIAL APPLICABILITY

The present invention can further improve the antitumor effect induced by photoimmunotherapy.

This application is based on a patent application No. 2021-171961 filed in Japan (filing date: Oct. 20, 2021), the contents of which are incorporated in full herein.

1. A method for treating cancer, comprising administering to a subject an effective amount of a complex of a photosensitive molecule and a molecule binding to a cancer-specific cell surface molecule, and an effective amount of an adjuvant excluding ligands for Toll-like receptor 3 (TLR3).

2. A method for treating cancer, comprising:
(a) administering to a subject an effective amount of a complex of a photosensitive molecule and a molecule binding to a cancer-specific cell surface molecule, wherein the complex is administered in combination with an effective amount of an adjuvant excluding ligands for Toll-like receptor 3 (TLR3), or
(b) administering to a subject an effective amount of an adjuvant excluding ligands for Toll-like receptor 3 (TLR3), wherein the adjuvant is administered in combination with an effective amount of a complex of a photosensitive molecule and a molecule binding to a cancer-specific cell surface molecule.

3. (canceled)

4. The method according to claim 1, wherein the adjuvant is a ligand for Toll-like receptor 9 (TLR9).

5. The method according to claim 4, wherein the ligand for Toll-like receptor 9 (TLR9) is a complex of a nucleic acid and a polysaccharide.

6. The method according to claim 5, wherein the nucleic acid is a nucleic acid comprising a type-K CpG oligodeoxynucleotide and polydeoxyadenylic acid.

7. The method according to claim 6, wherein the type-K CpG oligodeoxynucleotide is humanized.

8. The method according to claim 6, wherein the type-K CpG oligodeoxynucleotide comprises the nucleotide sequence shown in SEQ ID NO: 1.

9. The method according to claim 5, wherein the polysaccharide is β-glucan.

10. The method according to claim 9, wherein the β-glucan is schizophyllan or lentinan.

11. The method according to claim 1, wherein the adjuvant is a ligand for STING.

12. The method according to claim 11, wherein the ligand for STING is cGAMP.

13. (canceled)

14. The method according to claim 1, wherein the complex of a photosensitive molecule and a molecule binding to a cancer-specific cell surface molecule is administered intratumorally or intravenously, and the adjuvant is administered intratumorally or intravenously.

15. The method according to claim 1, wherein the complex of a photosensitive molecule and a molecule binding to a cancer-specific cell surface molecule, and the adjuvant are each administered intravenously.

16. The method according to claim 2, wherein the adjuvant is a ligand for Toll-like receptor 9 (TLR9).

17. The method according to claim 2, wherein the adjuvant is a ligand for STING.

18. A medicament for treating cancer, comprising a complex of a photosensitive molecule and a molecule binding to a cancer-specific cell surface molecule, and an adjuvant excluding ligands for Toll-like receptor 3 (TLR3).

19. The medicament according to claim 18, wherein the adjuvant is a ligand for Toll-like receptor 9 (TLR9).

20. The medicament according to claim 19, wherein the ligand for Toll-like receptor 9 (TLR9) is a complex of a nucleic acid and a polysaccharide.

21. The medicament according to claim 20, wherein the nucleic acid is a nucleic acid comprising a type-K CpG oligodeoxynucleotide and polydeoxyadenylic acid.

22. The medicament according to claim 18, wherein the adjuvant is a ligand for STING.
